SEQUENCE LISTING (15 sequences)

SEQ ID NO: 1 - DNA, 84 bp, Artificial Sequence
  Synthetic amplicon
ctaggtccgttcattaggacaaccctatcctagcgaactgtctgatcggctgagcatgggtcggaagagacatccgctaacggt

SEQ ID NO: 2 - DNA, 84 bp, Artificial Sequence
  Synthetic amplicon
gacggtcacagacctagagacgtactcccgatctgtgtcgatggacggaattagtgcgtacatctccctggtcggattctagag

SEQ ID NO: 3 - DNA, 84 bp, Artificial Sequence
  Synthetic amplicon
tgtgtcgggaatgttgacgtgtctgacactggtggaatacgcaacgcaagggccgcatgtgtccgcactagcgtagagtcttca

SEQ ID NO: 4 - DNA, 84 bp, Artificial Sequence
  Synthetic amplicon
acttggcgcgatgattgacaaaacaccgcggccgaaatcctttggcgtagtcctcgggtagttcggtcaaagttacagctggtt

SEQ ID NO: 5 - DNA, 84 bp, Artificial Sequence
  Synthetic amplicon
aaatgcgaggccgtgggatcgcgctgtatgcaccataccgtaaatgtccaaatacgcggtcgggggttgtaccggcaaatgtgc

SEQ ID NO: 6 - DNA, 84 bp, Artificial Sequence
  Synthetic amplicon
tggctggctaggcgagacataggtcaactggcttagcatacgcagctaataggctccgatgccgaatgcggatttaattccggg

SEQ ID NO: 7 - DNA, 84 bp, Artificial Sequence
  Synthetic amplicon
ttaaacgcacagtcacgtaggggtgagcacagttcgtccgactcccatcagcgcactcacatacggatgggtggtatcgggaag

SEQ ID NO: 8 - DNA, 84 bp, Artificial Sequence
  Synthetic amplicon
gaaccgggacctgagcccaaacgtcagtccgggctatatcaaatgagacgcacataaccgtccacccggcgtatatgcggatgc

SEQ ID NO: 9 - DNA, 84 bp, Artificial Sequence
  Synthetic amplicon
cagtgatgccgctacgtcggttaattgggattgcgacagcgtcgtcttgcagagcgatacgttccaaattgcgggtcctacagc

SEQ ID NO: 10 - DNA, 84 bp, Artificial Sequence
  Synthetic amplicon
accagctcctagagcgaattgcgctcagtgtaacgccgctacgcctctcgctcctgtaagccttatcggtggagggacttatac

SEQ ID NO: 11 - DNA, 84 bp, Artificial Sequence
  Synthetic amplicon
gacgtccgctccatcaacagcgacgacccgcataatgatcacgggacgctagatagctcgagttctcactctatgctctaggcc

SEQ ID NO: 12 - DNA, 86 bp, Artificial Sequence
  Synthetic amplicon
ggcacaaagaaatccagcgtcactaggtcagctaagccgaaaaatgtgtgcctgcgctcctcgcctcatctcgatgacatacgatg

SEQ ID NO: 13 - DNA, 3 bp, Artificial Sequence
  Primer
ttt

SEQ ID NO: 14 - DNA, 6 bp, Artificial Sequence
  Primer
cacaca

SEQ ID NO: 15 - DNA, 6 bp, Artificial Sequence
  Primer
gtgtgt

(19) United States
(12) Patent Application Publication
(10) Pub. No.: US 2005/0095634 A1
(43) Pub. Date: May 5, 2005

(54) qRT-PCR assay system for gene expression profiling

(75) Inventors: Joffre B. Baker, Montara, CA (US); Maureen T. Cronin, Los Altos, CA (US); Michael C. Kiefer, Clayton, CA (US); Xitong Li, Mountain View, CA (US); Kim Clark, Mountain View, CA (US)

Correspondence Address: HELLER EHRMAN WHITE & MCAULIFFE LLP, 275 MIDDLEFIELD ROAD, MENLO PARK, CA, 94025-3506, US

(73) Assignee: Genomic Health Inc., Redwood City, CA (US)

(21) Appl. No.: 10/963,286

(22) Filed: Oct. 11, 2004

Related U.S. Application Data
(60) Provisional application No. 60/512,556, filed on Oct. 16, 2003.

(51) Int. Cl. C12Q 1/68; C12P 19/34

(52) U.S. Cl. 435/6; 435/91.2

(57) ABSTRACT

The invention concerns an integrated, qRT-PCR-based system for analyzing and reporting RNA expression profiles of biological samples. In particular, the invention concerns a fully optimized and integrated multiplex, multi-analyte method for expression profiling of RNA in biological samples, including fixed, paraffin-embedded tissue samples. The gene expression profiles obtained can be used for the clinical diagnosis, classification and prognosis of various pathological conditions, including cancer.

### CROSS-REFERENCE TO RELATED APPLICATIONS

This is a non-provisional application filed under 37 CFR 1.53(b),claiming priority under USC Section 119(e) to provisional Application Ser. No. 60/512,556, filed Oct. 16, 2003.

### BACKGROUND OF THE INVENTION

1. Field of the Invention

The present invention concerns an integrated, qRT-PCR-based system for analyzing and reporting RNA expression profiles of biological samples. In particular, the invention concerns a fully optimized and integrated multiplex, multi-analyte method for expression profiling of RNA in biological samples, including fixed, paraffin-embedded tissue samples. The gene expression profiles obtained can be used for the clinical diagnosis, classification and prognosis of various pathological conditions, including cancer.

2. Description of the Related Art

In the past few years, several groups have published studies concerning the classification of various cancer types by microarray gene expression analysis [see, e.g. Golub et al., Science 286:531-537 (1999); Bhattacharjae et al., Proc. Natl. Acad. Sci. USA 98:13790-13795 (2001); Chen-Hsiang et al., Bioinformatics 17 (Suppl. 1):S316-S322 (2001); Ramaswamy et al., Proc. Natl. Acad. Sci. USA 98:15149-15154 (2001)]. Certain classifications of human breast cancers based on gene expression patterns have also been reported [Martin et al., Cancer Res. 60:2232-2238 (2000); West et al., Proc. Natl. Acad. Sci. USA 98:11462-11467 (2001)]. Most of these studies focus on improving and refining the already established classification of various types of cancer, including breast cancer. A few studies identify gene expression patterns that may be prognostic [Sorlie et al., Proc. Natl. Acad. Sci. USA 98:10869-10874 (2001); Yan et al., Cancer Res. 61:8375-8380 (2001); Van De Vivjer et al. New England Journal of Medicine 347: 1999-2009 (2002)], but due to inadequate numbers of screened patients, are not yet sufficiently validated to be widely used clinically.

The standard process for handling biopsy specimens has been, and still is, to fix tissues in formalin and then embed them in paraffin. Therefore, by far the most abundant supply of solid tissue specimens associated with clinical records is fixed, paraffin-embedded tissue (FPET). In the last decade several laboratories have demonstrated that it is possible to measure mRNA levels (i.e. gene expression) using FPET as a source of RNA [see, e.g. Rupp and Locker, Biotechniques 6:56-60 (1988); Finke et al., Biotechniques 14:448-453 (1993); Reichmuth et al., J. Pathol. 180:50-57 (1996); Stanta and Bonin, Biotechniques 24:271-276 (1998); Sheile and Sweeny, J. Pathol. 188:87-92 (1999); Godfrey et al., J. Mol. Diagn. 2:84-91 (2000); Specht et al., Am. J. Pathol. 158:419-429 (2001); and Abrahamsen et al., J. Mol. Diagn. 5:66-71 (2002)]. However, to date little evidence exists that DNA arrays can be effectively applied to FPE tissue RNA analysis (Karsten et al., Nucleic Acids Res. 30:E4 (2002)).

In order to further advance the use of gene expression analysis in clinical diagnosis and prognosis of various diseases, such as cancer, there is a great need for highly sensitive gene expression profiling approaches that enable simultaneous analysis of a large number of genes, using a small amount of biological sample. Especially in the field of cancer diagnosis and prognosis, it is essential for such methods to have the ability to analyze a wide range of gene expression levels, or any combination of genes, in an FPET sample, in a single gene expression profiling experiment.

### SUMMARY OF THE INVENTION

The present invention provides a highly sensitive and precise method that has multi-analyte capability and is suitable for the measurement of gene expression in aged, preserved, or processed tissue samples, such as fixed, paraffin-embedded (FPE) tissue samples.

In one aspect, the present invention concerns a method for determining RNA expression profile in a tissue sample comprising a plurality of RNA species, comprising the steps of:
(a) extracting RNA from the sample under conditions that provide a maximum representation of all transcribed RNA species present in the tissue sample;
(b) treating the RNA obtained with a reverse transcription mixture comprising a plurality of gene-specific oligonucleotides corresponding to at least a subset of said RNA species, dNTPs and a reverse transcriptase, under conditions allowing transcription of said RNA into complementary DNA (cDNA);
(c) quantitatively detecting each cDNA transcript,

wherein steps (a) and (b) are performed in separate reactions.

Optionally, the transcribed cDNA obtained in step (b) is amplified before performing step (c). Amplification can be performed in a variety of ways, including, for example, polymerase chain reaction (qPCR), in the presence of a set of forward and reverse primers to generate an amplicon, and a probe for each cDNA transcript.

The tissue can be a human tissue, including frozen or fixed, wax-embedded tissues.

The reverse transcription mixture in step (a) may comprise gene-specific oligonucleotides for at least about 10 RNA species, or at least about 15, or at least about 90, or at least 400, or at least about 800, or at least about 1600 RNA species.

The reverse transcription mixture may further comprise a plurality of random oligonucleotides, which are typically 6- to 1 0-nucleotide long. In a particular embodiment, at least in one of reverse transcriptase step (b) and qPCR amplification step, the number of oligonucleotides susceptible for self-priming or cross-priming is minimized, for example by a computer algorithm.

In another embodiment, the reverse transcription mixture comprises RNA of at least one, and usually about 5 to about 10 normalization reference sequences. In a further embodiment, each qRT-PCR reaction includes at least one internal calibration reference sequence. Preferably, one or more of the internal calibration reference sequences include sequences which have no significant homology to any sequence in the human genome.

The tissue sample can, for example, be a frozen or fixed, such a formalin-fixed, paraffin-embedded (FPE) biopsy sample from a tumor, e.g. a cancer. Other forms of tissue samples include, without limitation, ethanol-fixed tissues and tissues fixed by variations of the traditional formalin and/or ethanol fixation methods, flash frozen, OCT (Optimal Cutting Temperature compound) frozen, and fresh tissue samples, and the like. Typical cancers include, without limitation, breast cancer, colon cancer, lung cancer, prostate cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, and brain cancer.

In a particular embodiment, the cancer tissue comprises fragmented RNA, where the gene target amplicons can be less than about 100 nucleotides long, or less than about 90 nucleotides long, or less than about 80 nucleotides long.

In another embodiment, the difference between the length of the amplicons of the target genes and the reference genes is not more than about 15%, or less than about 10%.

The gene expression levels can be normalized relative to the normalization reference sequence or sequences, where suitable normalization reference genes include, for example, β-ACTIN, CYP1, GUS, RPLPO, TBP, GAPDH, and TFRC.

In a further embodiment, the gene expression levels are corrected relative to one or more universal internal calibration reference sequences.

The method of the present invention may further include the step of identifying one or more genes the expression of which is correlated with the presence or likelihood of recurrence of cancer, or the likelihood of responding to a chemotherapeutic drug or drug set, and optionally the further step of subjecting the gene expression profile to statistical analysis.

In a further embodiment, the method further includes the step of preparing a report for a subject whose cancer tissue is analyzed, which may include a statement of likelihood of survival without cancer recurrence, or likelihood of response to a certain chemotherapeutic drug or drug set.

In another aspect, the invention concerns a kit that includes one or more of the following components: extraction buffer/reagents and protocol; reverse transcription buffer/reagents (including pre-designed primers) and protocol; qPCR buffer/reagents (including pre-designed probes and primers) and protocol; data retrieval and analysis software.

Further details of the individual steps are discussed below.

### BRIEF DESCRIPTION OF THE DRAWINGS

FIG. 1. Flow chart of the gene expression profiling method of the invention.

FIG. 2. Size distribution of FPE Tissue RNA from 12 tumor specimens. Total RNA was extracted from breast cancer specimens as described in Example 1. One μl from each RNA extract (1/30 of the sample) was analyzed using an Agilent 2100 Bioanalyzer, RNA 6000 Nanochip. Lanes 1-4, 5-8, and 9-12 contain RNA from samples archived about one, six and 17 years, respectively. Lanes M1 and M2 contain two different sets of molecular weight marker RNA (sizes denoted in bases).

FIG. 3. Expression ranges for 92 genes in 62 breast cancer specimens. TaqMan qRT-PCR was used to measure mRNA levels as described in Example 1, and expression relative to six reference genes. The mean and mean standard deviation of the expression values across all tested patients is shown for each gene. Each box represents the mean mRNA level for all tested tumor specimens, and the error bars indicate the standard deviation of all measurements for that gene. Expression values (Y-axis) are normalized relative to reference genes expressed as log base 2 (log₂) values. Normalized mRNA levels of test genes are defined as 2^{ΔCT}+10.0, where Δ C_{T}=C_{T} (mean of six reference genes)−C_{T} (test gene).

FIGS. 4A-B. Mean C_{T} (cycle threshold) values for 92 genes in 62 patient samples as a function of paraffin block archive storage time. The X axis shows the year each specimen was archived. The Y axis shows mean expression values for all tested genes. Each symbol represents a separate patient. Panel 4A: Raw mean C_{T} expression values for all specimens. Panel 4B: Expression values after normalization relative to six reference genes. Normalized mRNA levels are as defined in the legend to FIG. 3 above. Reference genes were β-ACTIN, CYP1, GUS, RPLPO, TBP, and TFRC. Solid lines: linear regression best fit.

FIG. 5. Flow chart for a program to identify oligonucleotide sequences likely to self-prime or cross-prime.

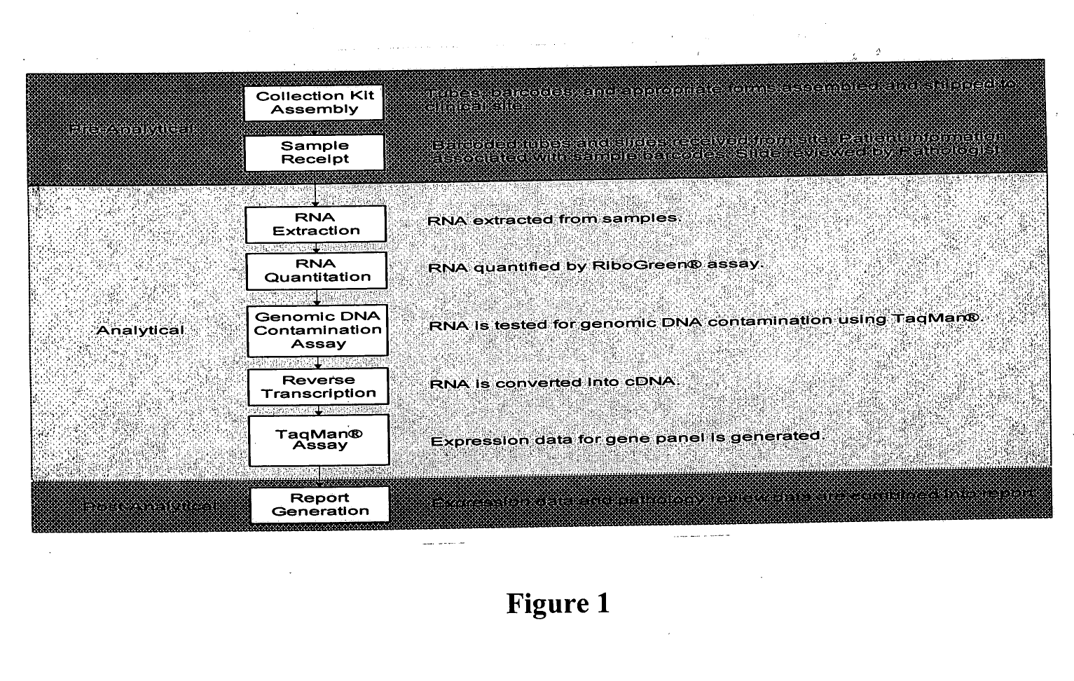

### DETAILED DESCRIPTION OF THE PREFERRED EMBODIMENT

A. Definitions

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, NY 1994), and March, Advanced Organic Chemistry Reactions, Mechanisms and Structure 4th ed., John Wiley & Sons (New York, N.Y. 1992), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

The term “gene expression profiling” is used in the broadest sense, and includes methods of quantification of mRNA and/or protein levels in a biological sample.

The term “microarray” refers to an ordered arrangement of hybridizable array elements, preferably polynucleotide probes, on a substrate.

The term “polynucleotide,” when used in singular or plural, generally refers to any polyribonucleotide or polydeoxribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes cDNAs. The term includes DNAs (including cDNAs) and RNAs that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritiated bases, are included within the term “polynucleotides” as defined herein. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The term “oligonucleotide” refers to a relatively short polynucleotide, including, without limitation, single-stranded deoxyribonucleotides, single- or double-stranded ribonucleotides, RNA:DNA hybrids and double-stranded DNAs. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

The terms “differentially expressed gene,” “differential gene expression” and their synonyms, which are used interchangeably, refer to a gene whose expression is activated to a higher or lower level in a subject suffering from a disease, specifically cancer, such as breast cancer, relative to its expression in a normal or control subject. The terms also include genes whose expression is higher or lower level at different stages of the same disease. The terms also include genes whose expression is higher or lower in patients who are significantly sensitive or resistant to certain therapeutic drugs. It is also understood that a differentially expressed gene may be either activated or inhibited at the nucleic acid level or protein level, or may be subject to alternative splicing to result in a different polypeptide product. Such differences may be evidenced by a change in mRNA levels, surface expression, secretion or other partitioning of a polypeptide, for example. Differential gene expression may include a comparison of expression between two or more genes or their gene products, or a comparison of the ratios of the expression between two or more genes or their gene products, or even a comparison of two differently processed products of the same gene, which differ between normal subjects and subjects suffering from a disease, specifically cancer, or between various stages of the same disease. Differential expression includes both quantitative, as well as qualitative, differences in the temporal or cellular expression pattern in a gene or its expression products among, for example, normal and diseased cells, or among cells which have undergone different disease events or disease stages, or cells that are significantly sensitive or resistant to certain therapeutic drugs For the purpose of this invention, “differential gene expression” is considered to be present when there is at least an about two-fold, preferably at least about four-fold, more preferably at least about six-fold, most preferably at least about ten-fold difference between the expression of a given gene in normal and diseased subjects, or in various stages of disease development in a diseased subject, or in patients who are differentially sensitive to certain therapeutic drugs.

The phrase “gene amplification” refers to a process by which multiple copies of a gene or gene fragment are formed in a particular cell or cell line. The duplicated region (a stretch of amplified DNA) is often referred to as “amplicon.” Frequently, the amount of the messenger RNA (mRNA) produced, i.e., the level of gene expression, also increases in proportion to the number of copies made of the particular gene.

The term “prognosis” is used herein to refer to the prediction of the likelihood of cancer-attributable death or progression, including recurrence, metastatic spread, and drug resistance, of a neoplastic disease, such as breast cancer.

The term “prediction” is used herein to refer to the likelihood that a patient will respond either favorably or unfavorably to a drug or set of drugs, and also the extent of those responses, or that a patient will survive, following surgical removal or the primary tumor and/or chemotherapy for a certain period of time without cancer recurrence. The predictive methods of the present invention are valuable tools in predicting if a patient is likely to respond favorably to a treatment regimen, such as surgical intervention, chemotherapy with a given drug or drug combination, and/or radiation therapy, or whether long-term survival of the patient, following surgery and/or termination of chemotherapy or other treatment modalities is likely.

The term “long-term” survival is used herein to refer to survival for at least 5 years, more preferably for at least 8 years, most preferably for at least 10 years following surgery or other treatment.

The term “tumor,” as used herein, refers to all neoplastic cell growth and proliferation, whether malignant or benign, and all pre-cancerous and cancerous cells and tissues.

The terms “cancer” and “cancerous” refer to or describe the physiological condition in mammals that is typically characterized by unregulated cell growth. Examples of cancer include but are not limited to, breast cancer, colon cancer, lung cancer, prostate cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, and brain cancer.

The “pathology” includes all phenomena that compromise the well-being of the patient. In the case of cancer (tumor), this includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc.

The term “normalization reference sequence” is used herein to refer to a genomic DNA sequence that is transcribed at a relatively constant level within different individuals, different tissues, and different tissue environments, and can be used as a control for variability in amounts and quality of RNA in different specimens, thereby allowing comparison of gene expression profiles between different patients and specimen samples.

The term “internal calibration reference sequence” refers to oligonucleotide sequences that can be used as inert internal assay performance calibration controls since they do not represent sequences expressed in the human genome. These universal “inert” assays can act as internal controls for process calibration by virtue of the fact their components are synthetic and the resulting qRT-PCR reactions serve the purpose of monitoring a consistent assay performance baseline against which accompanying biologically informative assays may be compared. These calibrator sequences and their primers and probes can be constructed and combined to yield a consistently predictable assay outcome under standard assay conditions. This baseline performance by inference may be extrapolated to assays run under the same conditions in the same reaction volume or well. Deviation from expected values provides a measure of parallel deviation occurring in the biologically informative assays. That is, ideally, if one of these reactions is added at a standard primer and probe concentration with a known template concentration, the reaction C_{T} should be predictable 100% of the time. When a deviation from the expected result occurs, it can be assumed that reaction inhibition or reagent malfunction has occurred.

B. Detailed Description

The practice of the present invention will employ, unless otherwise indicated, conventional techniques of molecular biology (including recombinant techniques), microbiology, cell biology, and biochemistry, which are within the skill of the art. Such techniques are explained fully in the literature, such as, “Molecular Cloning: A Laboratory Manual”, 2^{nd} edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology”, 4^{th} edition (D. M. Weir & C. C. Blackwell, eds., Blackwell Science Inc., 1987); “Gene Transfer Vectors for Mammalian Cells” (J. M. Miller & M. P. Calos, eds., 1987); “Current Protocols in Molecular Biology” (F. M. Ausubel et al., eds., 1987); and “PCR: The Polymerase Chain Reaction”, (Mullis et al., eds., 1994).

The present invention provides an optimized, quality-controlled high throughput system for analyzing and reporting RNA expression profiles in biological patient samples. The method of the present invention is particularly suitable to analyze biological samples containing poor quality, fragmented or chemically modified RNA, including aged, preserved and/or processed samples, such as, for example, samples of fixed, paraffin-embedded (FPE) tissues, forensic and pathology samples. Expression profiling by this analytical method is not limited by the sequence of the target gene, and can be applied to specifically analyze any gene or combination of genes expressed in biological samples including biological samples containing poor quality, fragmented or chemically modified RNA, such as FPE tissue samples. Indeed, there is no upward boundary on the multiplicity of gene targets that can be included in the expression profile analysis of a single fixed paraffin-embedded sample.

The quantitative RT-PCR (qRT-PCR) gene expression profiling system of the invention includes several strategies in the RNA extraction, reverse transcription, cDNA amplification, data processing and analysis steps, which improve quality, efficiency, gene scalability and biological sample conservation. Some of these steps are detailed below.

(1) RNA extraction requires tissue disruption, nuclease inactivation, hydrolysis of genomic DNA, and selective recovery of RNA. The present invention includes a highly effective protocol for RNA extraction from FPE tissues, including the use of a new tissue lysis buffer, and improvements in the way the remaining protein is precipitated following lysis.

(2) Reverse transcription (RT) is carried out with gene-specific primers that also serve as the reverse primers for the later cDNA amplification step. Typically, reverse transcription is carried out using oligo-dT priming. However, because extracted FPE RNA may be highly fragmented, most of the mRNA sequences obtained from such source may be separated from polyA tails, and therefore not accessible for reverse transcription via oligo-dT priming. In order to overcome the problems associated with RNA fragmentation, random hexamers are commonly used for priming cDNA synthesis. The present invention demonstrates that gene-specific priming is possible, more efficient than random hexamer priming, and can be used efficiently despite the extensive fragmentation of the FPET RNA

(3) In the process of the present invention, the gene-specific primer used in the RT step also serves as the reverse primer for the cDNA amplification step. This is to our knowledge the most efficient priming strategy for FPE tissue RNA. If the primer used for the RT step is not identical to the reverse primer used in the cDNA amplification step, assay sensitivity decreases as a result of increasing probability that the created cDNA sequence does not extend completely through the amplicon sequence due to (i) the limited length of the RNA and (ii) the presence of formalin-modified bases.

(4) The RT and cDNA amplification steps are carried out as a two-stage process. This enables the respective enzymatic reactions (reverse transcriptase and Taq polymerase in the case of TaqMan® PCR) to be carried out at each enzyme's optimal conditions, such as enzyme, dNTP and primer concentrations, temperature, buffer and pH. This feature further increases the sensitivity of the assay.

(5) The RT step is multiplexed, specifically by combining in one reaction a large number of reverse primers, typically up to 96, or even 768 genes. This provides a practical method for a multi-analyte assay. The alternative of carrying out the RT reaction with one reaction per gene would require measurement of prohibitively small liquid volumes or the use of much greater amounts of expensive RT enzyme and valuable patient biopsy specimens. Accordingly, the multiplexed RT step in the process of the invention provides optimal sample conservation while still maintaining maximum analytical sensitivity for multi-analyte assay of gene expression. The protocol includes use of multiplexed gene-specific primer pools for the genes to be profiled, which can be also combined with random oligonucleotide priming (hexamers to decamers in most cases).

(6) The qPCR step can also be multiplexed, as needs be, to permit assay of more than one, typically up to three, mRNA species per reaction, although larger numbers are also possible. Just as in the RT step, multiplexing preserves patient biopsy specimen and permits simultaneous assay of greater numbers of mRNA species thereby increasing the efficiency screening power of the entire process.

(7) A component of the multiplexing steps (i.e. steps (5) and (6) above) is incorporation into primer and probe design a program to check oligonucleotide cross-priming- and self-priming. Cross-priming or self-priming occur when the 3′ region of an oligonucleotide is complementary to and base pairs with another oligonucleotide or itself. With a perfect match of over 5 bases, cross-priming or self-priming is relatively likely, and the probability increases with increasing match length. Because in the process of the present invention multiple different oligonucleotide primers and probes are present in the same reaction volume, cross-priming or even self-priming might happen, leading to an undesired polymerization, increase in reaction background noise, and decrease in target signal. The program incorporated into the process of the invention helps eliminate the artifacts associated with cross-priming and self-priming.

(8) Finally, the method of the present invention employs unique normalization strategies and allows the use of universal reference gene primers/probes to maximize sensitivity, reliability and sample to sample comparability.

As a result of the unique steps included in the gene expression profiling method of the present invention, the method herein provides improved sensitivity and efficiency, while using minimized amounts of the RNA sample analyzed. Typically, as little as 5 μl reaction volume (using 0.8-1.0 ng of FPE tissue RNA/qPCR reaction well) can be used for analysis by the method of the present invention. Further experiments have shown that even as small as 2.5 μl reactions can be successfully used, containing 0.25-1.0 ng FPE RNA equivalent (cDNA) per reaction well. This unique sequence of steps has the additional advantage that it results in multianalyte assay panels with internally consistent performance and low analytical “noise” making them useful as clinical diagnostic panels.

RNA Extraction and Purification

The first analytical step of the gene expression profiling method of the present invention is the extraction and purification of RNA to be analyzed from biological samples. The starting material can, for example, be total RNA isolated from human tumors or tumor cell lines, and corresponding normal tissues or cell lines, respectively. Thus RNA can be isolated from a variety of primary tumors, including breast, lung, colon, prostate, brain, liver, kidney, pancreas, spleen, thymus, testis, ovary, uterus, head and neck, etc., tumor, or tumor cell lines. If the source of mRNA is a primary tumor, mRNA can be extracted, for example, from frozen or archived paraffin-embedded fixed (e.g. formalin-fixed) tissue samples (FPET). If the RNA source is from FPET, this method includes the removal of paraffin. It is well known that deparaffinization of FPE tissues can be accomplished by protocols employing xylenes as a solvent. Alternatively, RNA can be extracted and purified using a protocol in which dewaxing is performed without the use of any organic solvent, thereby eliminating the need for multiple manipulations associated with the removal of the organic solvent, and substantially reducing the total time to the protocol. According to this alternative protocol, wax, e.g. paraffin, is removed from wax-embedded tissue samples by incubation at 65-75° C. in a lysis buffer that solubilizes the tissue and hydrolyzes the protein, followed by cooling to solidify the wax. For further details see, for example, co-pending application Ser. No. 10/388,360 filed on Mar. 12, 2002, and International Application PCT/US 03/07713 filed on Mar. 12, 2003, the entire disclosures of which are hereby expressly incorporated by reference. A complete protocol for extraction of RNA from FPE tissue is shown in Example 3. A key step in the process is effective extraction of the RNA from the tissue. We have discovered a highly effective extraction buffer for FPE tissue, which consists of 330 μg/ml proteinase K, 4M urea, 10 mM TrisCl, pH 7.5, and 0.5% sodium lauroyl sarcosine. After extraction, the RNA is then incubated with DNase 1 by standard methods, to remove DNA. The method described in Example 3, in particular the use of the described extraction buffer and protocol, results in recovery of a representation of the transcribed RNA species present in a tissue down to oligonucleotide sizes below 60 bases in length. The method includes, but is not limited to, quantitative recovery of ribonucleic acids of a particular size distribution as well as quantitative recovery of selected specific RNA sequences, longer than a specified minimum length based on specific affinity or hybridization capture techniques.

One method of accomplishing quantitative recovery of all purified nucleic acids is to use carrier-mediated precipitation of the purified material. Alternatively, chromatographic or affinity capture and release based methods may be used to recover selective fractions of the purified nucleic acid. These methods may include a variety of membranes or matrices with size exclusion properties or affinity membranes or matrices requiring prior modification of the purified nucleic acid with a hapten or “capture nucleotide sequence”. These types of purification rely on a pretreatment modification step to generically modify all ribonucleic acids in a sample generically in such a way as to enable quantitative ribonucleic acid recovery from a tissue sample.

Since the method of the present invention is not restricted to RNA-specific assays (designs spanning an intron), it is desirable to include a step to ensure that DNA contamination of the purified RNA is kept below a threshold above which the presence of genomic DNA would compromise accurate qRT-PCR measurement of mRNA species in a panel. RNA extracts that still have genomic DNA above a certain threshold need to be retreated with DNase, so that the qRT-PCR assay only reports RNA signals, not DNA signals.

2. General Description of Quantitative PCR for Residual Genomic DNA

Since many qRT-PCR assays are not designed to include intron splice junctions and may be susceptible to quantitation errors if significant amounts of genomic DNA are present in a sample extract, it is common practice to run control reactions in parallel with qRT-PCR reactions to measure or estimate this effect. One common way to do construct a control is to include a parallel reaction to the qRT-PCR reaction in which reverse transcription has not been done. The assumption is that any positive result from this reaction is due to genomic DNA template. Unfortunately, in the absence of specific reverse transcribed template the RT negative or “no-RT” control can be subject to sporadic artifacts that appear to be positive reactions but actually represent artifactual primer and probe interactions with each other and with the RNA in the reaction solution. A preferred approach to control for the presence of significant residual genomic DNA in a sample extract would be to pre-qualify an RNA extract as “genomic DNA free” to the extent it will not give measurable interference in any qRT-PCR assay. Such an approach would be satisfied by designing a sensitive qPCR assay specific for genomic DNA. The attributes of the ideal assay would include: an amplicon (assay target template) design that is redundant in the unexpressed genome, preferably on multiple chromosomes; the redundancy should be at a high enough multiplicity that the assay sensitivity would be essentially unaffected by the chromosomal deletions and duplications that are common in cancer; the qPCR assay design should be of very high efficiency and sensitivity to a very low concentration of input genomic DNA. This assay would be used to screen purified RNA to qualify it for qPCR and provides the following advantages: 1) it preserves RNA sample since a parallel control for each gene in an expression screen would not be required; 2) it simplifies interpretation of the result since a single assay with a stringently defined threshold will eliminate the need to interpret variable and sporadic results that come from “no-RT” controls that are not tested for genomic DNA sensitivity and 3) it provides for sample qualification prior to commitment to qRT-PCR, eliminating the potential waste of a sample that has significant residual genomic DNA where the qRT-PCR cannot be interpreted. Examples of sensitive genomic DNA qPCR assays include a β-actin (NM_{—}001101) assay defined by a target template amplicon present on at least 7 chromosomes with near perfect identity, and an RPLPO (NM_{—}001002) assay defined by a target template amplicon present on 5 chromosomes with near perfect identity.

3. General Description of Reverse Transcriptase PCR

Reverse transcription PCR (qRT-PCR) is perhaps the most sensitive and flexible gene expression profiling method, which can be used to compare mRNA levels in different sample populations, in normal and diseased, e.g. tumor, tissues, with or without drug treatment, to characterize patterns of gene expression, to discriminate between closely related mRNAs, and to analyze RNA structure.

As RNA cannot serve as a template for PCR, the first step in gene expression profiling by qRT-PCR is the reverse transcription of the RNA template into cDNA, followed by its exponential amplification in a PCR reaction. The two most commonly used reverse transcriptases are avian myeloblastosis virus reverse transcriptase (AMV-RT) and Moloney murine leukemia virus reverse transcriptase (MMLV-RT). The reverse transcription step is typically primed using gene specific primers, random hexamers, or oligo-dT primers, depending on the circumstances and the goal of expression profiling. For example, extracted RNA can be reverse-transcribed using a GeneAmp® RNA PCR kit (Perkin Elmer, Calif., USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase, which has a 5′-3′ exonuclease activity but lacks a 3′-5′ proofreading endonuclease activity. Thus, TaqMan® PCR typically utilizes the 5′ exonuclease activity of Taq or Tth polymerase to hydrolyze a fluorescently-labelled hybridization probe bound to its target amplicon, but any enzyme with equivalent 5′ exonuclease activity can be used. Two oligonucleotide primers are used to generate an amplicon typical of a PCR reaction. A third oligonucleotide, or probe, is designed to hybridize to a nucleotide sequence located between the two PCR primers. The probe is non-extendible by Taq DNA polymerase enzyme, and is 5′ labeled with a reporter fluorescent dye and a 3′ labeled with a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second chromophore. One molecule of reporter dye is liberated for each new molecule synthesized, and detection of the unquenched reporter dye provides the basis for quantitative interpretation of the data.

qRT-PCR can be performed using commercially available equipment, such as, for example, ABI PRISM 7900™ Sequence Detection System™ (Perkin-Elmer-Applied Biosystems, Foster City, Calif., USA), or LightCycler® (Roche Molecular Biochemicals, Mannheim, Germany). In a preferred embodiment, the 5′ exonuclease procedure is run on a real-time quantitative PCR device such as the ABI PRISM 7900™ Sequence Detection System™ or one of the similar systems in this family of instruments. The system consists of a thermocycler, laser, charge-coupled device (CCD), camera and computer. The system amplifies samples in 96-well or 384 well formats on a thermocycler. During amplification, laser-induced fluorescent signal is collected in real-time through fiber optic cables for all reaction wells, and detected at the CCD. The system includes software for running the instrument and for analyzing the data.

Exonuclease assay data are initially expressed as C_{T}, or the threshold cycle, values. As discussed above, fluorescence values are recorded during every PCR cycle and represent the amount of released fluorescent probe, which is directly proportional to product amplified to that point in the amplification reaction. The point when the fluorescent signal is first recorded as statistically significant is the threshold cycle (C_{T}).

To minimize errors and the effects of sample-to-sample variation and process variability, qRT-PCR is usually performed using an internal reference standard. The ideal internal standard is a set of transcribed sequences, “normalization reference sequences”, that are expressed at a relatively constant level among different patients or subjects, and are unaffected by the experimental treatment. RNAs frequently used to normalize patterns of gene expression include, among others, are mRNAs for glyceraldehyde-3-phosphate-dehydrogenase (GAPDH) and β-actin.

qRT-PCR is compatible both with quantitative competitive PCR assays in which an internal competitor for each target sequence is used for normalization, and with quantitative comparative PCR assays using a normalization gene or genes contained within the sample, as a gene for qRT-PCR normalization referencing. For further details see, e.g. Held et al., Genome Research 6:986-994 (1996).

The steps of a representative protocol for profiling gene expression using fixed, paraffin-embedded tissues as the RNA source, including RNA isolation, elimination of residual genomic DNA, and PCR amplification are given in various published journal articles (for example: Godfrey et al. supra and Specht et al., supra). Briefly, a representative process starts with cutting about three 10 μm thick sections of paraffin-embedded tumor tissue samples. The RNA is then extracted, and protein and DNA are removed. After analysis of the RNA concentration, RNA repair and/or amplification steps may be included, if necessary, and RNA is reverse transcribed using gene-specific primers followed by PCR.

4. Improvements in the qRT-PCR Protocol

As discussed above, the method of the present invention includes significant improvements in several steps of the standard qRT-PCR protocol, including the use of gene-specific primers in combination with random oligomer primers in a multiplex RT step, using the gene specific primer used in the RT step as the reverse primer in the subsequent cDNA amplification step, separation of the RT and cDNA amplification steps, primer and probe design, which includes selecting designs optimized to perform similarly (enabling their values to be compared across a sample) multiplexing, new normalization strategy and analysis of the data obtained. These improvements have been summarized above, and will be discussed in greater detail below.

(a) Simultaneous Analysis of a Plurality of Genes

As noted before, both the RT and the PCR step of the present invention may be multiplexed, i.e. performed by analyzing a plurality of genes in the same reaction. Thus, the reverse transcription mixture can include primers for a large number of genes. While, for instrumentation compatibility, primers for 96 genes are often included in the reaction mixture at the RT step, the method is not so limited. Multiplexing of the RT step can be successful using primers for up to 400, or up to 800, or even up to 1600 different genes in one reaction mixture. Similarly, the PCR step may be multiplexed, i.e. may include a plurality of genes in the same reaction for amplification.

In a particular embodiment of the method of the present invention, sets of optimized PCR primers and detection probes are combined, where each reaction contains multiple PCR primers and detection probes, specific for up to 5 different cDNAs, or combinations of cDNAs and internal calibrators.

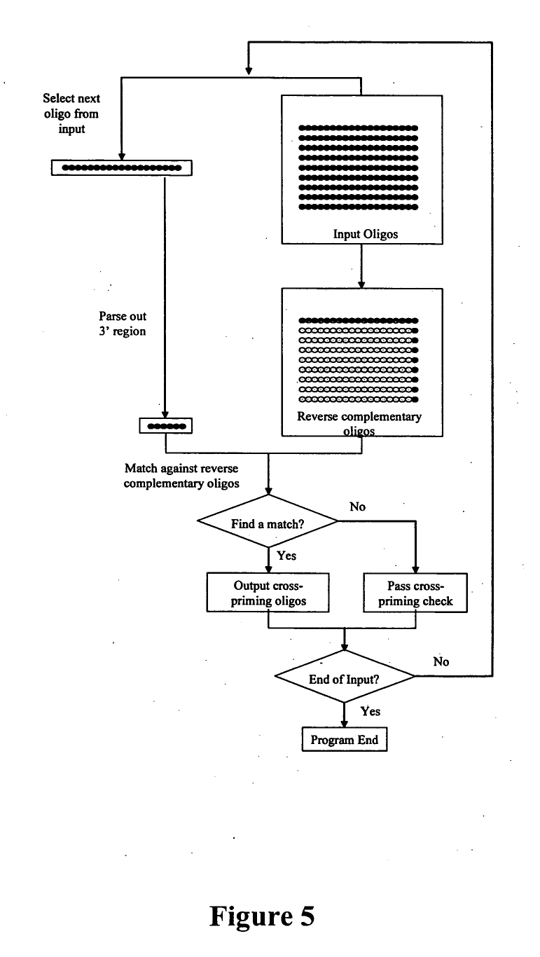

All primers and probes in this module have been globally optimized, in part via application of the self-priming and cross-priming check software program that is portrayed by the flowchart shown in FIG. 5. Optimized primers and probes behave similarly under a single set of homogenous assay conditions without non-specific interaction to form non-specific PCR products or primer dimer species and where the reverse PCR primer for each gene in the panel is substantially the same as the reverse transcription primer used to generate the cDNA in the prior reverse transcription step. It is also important that the residual genomic DNA content be kept below a threshold level which can be tolerated by the qRT-PCR assay of the present invention. The detection of each gene product during PCR can be performed by using any of the standard forms of signal detection in molecular assays using fluorescence, mass spectrometry, etc.

(b) Primer and Probe Design

The reverse transcription reaction and subsequent PCR amplification are performed with a pool of gene specific primers with or without additional random oligomers, typically random hexamers to decamers (which can incrementally increase sensitivity of the assay).

If FPE tissue samples, or other aged, preserved or processed samples, are analyzed, the extracted RNA tends to be fragmented, and amplicon sizes are preferably limited to less than about 100 bases, more preferably less than about 90 bases, even more preferably less than about 80 bases in length.

The primers and probes are typically designed following well known principles. Thus, for example, primers or probes that span intron-exon splice junctions are preferred. Generally, primers that have 3′ ends with strings of homopolymer or tandem repeat nucleotide sequences, such as TTT (SEQ ID NO: 13), CACACA (SEQ ID NO: 14), GTGTGT (SEQ ID NO: 15), should be avoided, unless there are absolutely no other high quality primer or probe candidates. The 5′ end of the probe should be at least one nucleotide away from the 3′ end of the primer that shares the same template strand. Probes that have a 5′ G should be avoided. The reverse complementary strand of probes that contain more G's than C's should be used unless they have a 5′ G. In the latter case, the forward strand should be used as the probe, The strand containing 5′G should never be used. These rules should be hierarchical, with Tm and priming efficiency weighing more heavily than sequence composition considerations. An example of a useful method reference for primer and probe design is: Rosen, S. and Skaletsky H. J. Primer3 on the WWW for general users and for biologist programmers. Krawetz, S., Misener, S., (eds.) Bioinformatics Methods and protocols: Methods in Molecular Biology, 365-386. 2000. Totowa, N.J., Humana Press.

A critical part of the protocol for selection of probe/primer sets for use with FPET RNA is empirical testing. For each gene of interest, preferably three different probe/primer sets are designed, synthesized, and tested for primer dimers using the SYBR Green Assay (Applied Biosystems, Inc.). Sets with primer dimers are excluded. Next, probe primer sets are tested using full length high quality RNA and FPE fragmented RNA templates. Criteria for probe/primer selection include sensitivity (low C_{T}), signal to noise (greatest ΔRn), reproducibility (lowest standard deviation between replicate reactions), and linearity of response to input target concentration.

(c) Control of Self- and Cross-Priming of Oligonucleotides

As discussed earlier, to improve the throughput and efficiency of the gene expression profiling method of the invention, preferably multiple oligonucleotides are used in one reaction (multiplexing). Thus, for example, a multiplexed qPCR reaction usually contains several sets of oligos, each set being composed of two PCR primers and a probe. Similarly, the RT step of the process typically employs a pool of gene-specific primers. In both steps, it is important to prevent the self-priming and cross-priming activity among the oligonucleotides present in order to achieve unbiased results. As part of the improved gene expression profiling system herein, an algorithm has been developed and implemented as a Per1 program to minimize cross-priming and self-priming of oligonucleotide primers and probes in multiplexed reactions. The algorithm for this is illustrated in FIG. 5. Briefly, the 3′ region for each oligonucleotide from the input is examined against all oligonucleotides present in the reverse complementary pool, and matches are identified. If there is a match, then it will output the self- or cross-priming oligonucleotides (both priming and target oligos). If there is no match, then the input passes the self- or cross-priming check.

(d) Normalization Strategy

To be able to compare qRT-PCR data from different tissue specimens, it is necessary to correct for relative differences in input RNA quantity and quality. Such differences arise primarily from the variability inherent in processing surgical tissue specimens, including relative mass of tissue, the time between surgery and formalin fixation, and the storage time after fixation. Further variability might result from differences in the methods and/or reagents used for tissue fixation, and storage time following fixation. A further consideration is the cumulative variability accrued while processing each sample from RNA extraction through quantitation, reverse transcription to cDNA and PCR. This correction is accomplished by normalizing raw expression values relative to a set of genes that vary little in their median expression among different tissue specimens (“normalization reference genes”). It has been demonstrated that following the process of the present invention, including the normalization strategy used, RNA extracted from a variety of sources, using variety of fixative protocols and reagents can be analyzed successfully.

The use of RNA from FPE tissues for gene expression profiling introduces an additional element of variability into qRT-PCR analysis. It is well known that RNA extracted from FPE tissue specimens is often present as fragments less than about 300 bases in length. Since FPE tissues are the most widely available clinical samples, any qRT-PCR based diagnostic or prognostic method must address specific issues associated with the poor quality and variability of FPE RNA.

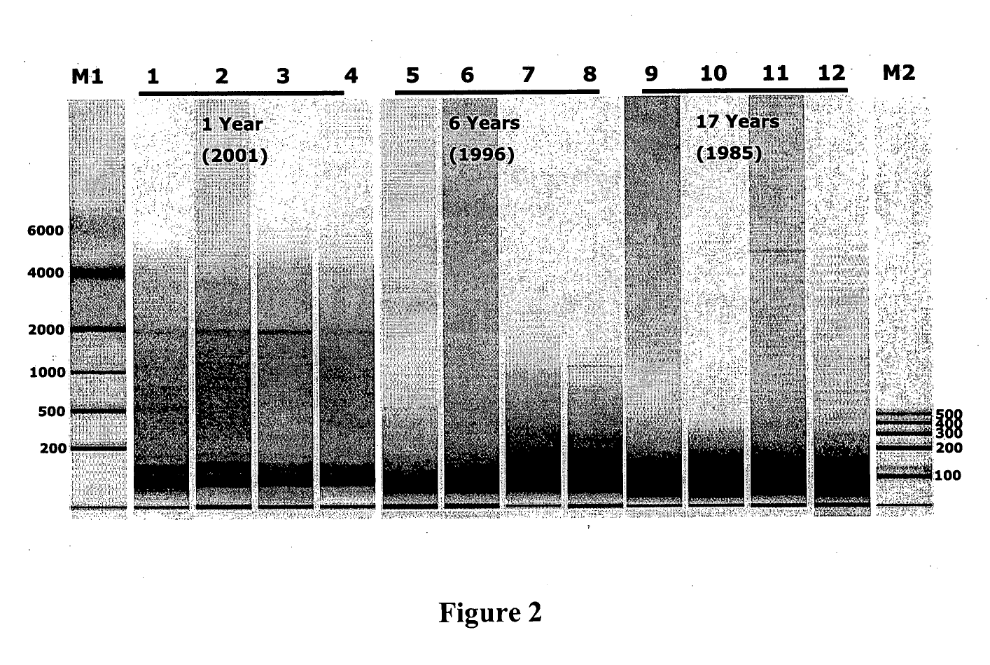

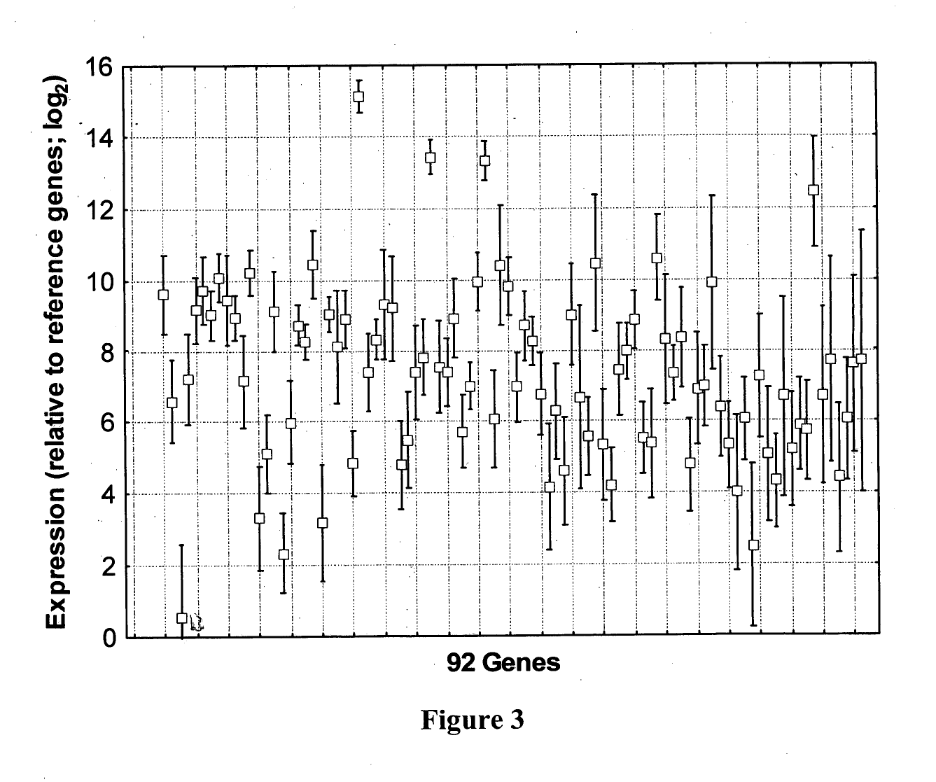

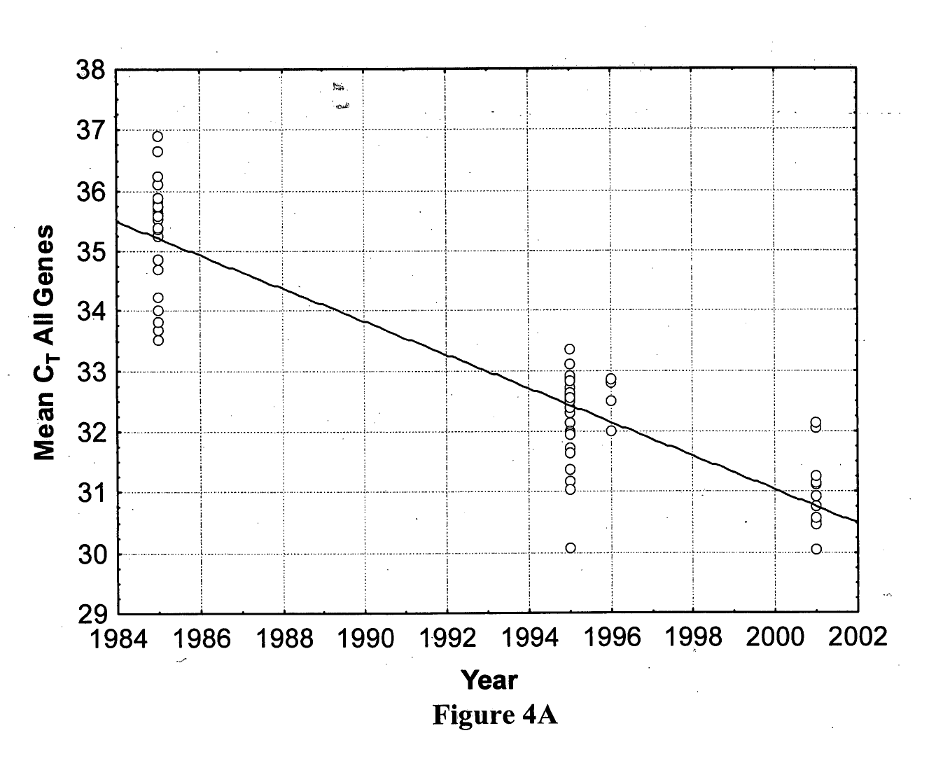

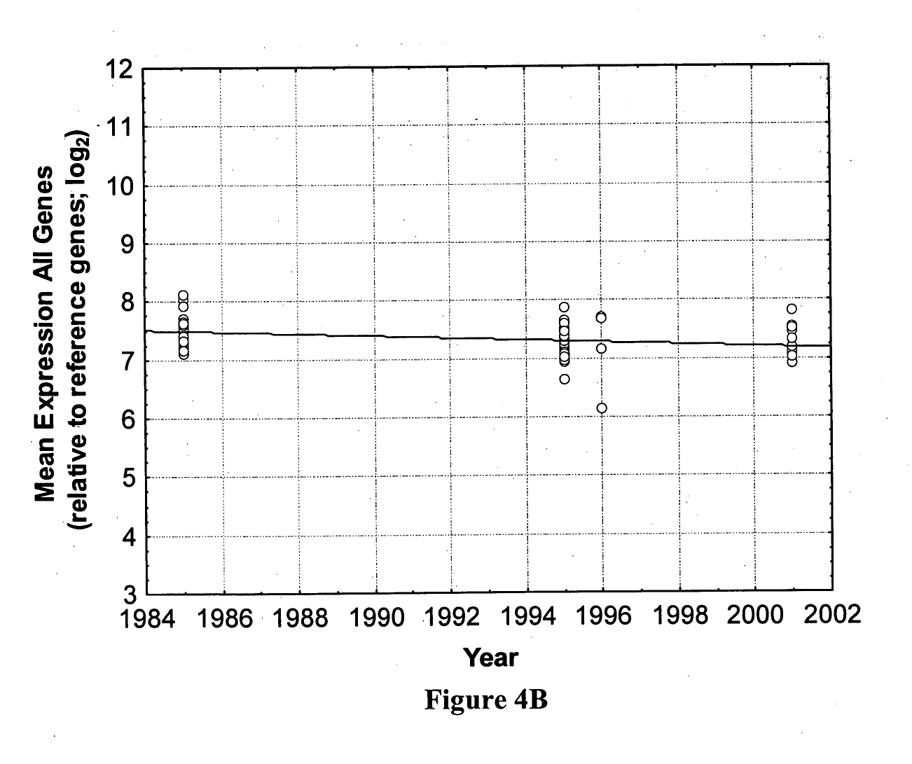

The present inventors have observed that RNA in FPE tissue specimens continues to degrade with increased storage time, and that this degradation results in a marked decline of mRNA assay signal strength (see FIGS. 2 and 4). Based on this observation and related experimental data showing that the rate of RNA strand breakage is proportionate with the length of amplicon (FIG. 4A), it has been found that the length of normalization reference gene amplicons used for normalization is critical for the accuracy and reliability of gene expression data. The breakdown of RNA strands in FPE tissue samples during storage is random. If the reference gene amplicon is too short, relative to the lengths of the test genes in the assay panel, the level of the target genes is underestimated.

Similarly, if the reference gene amplicon is too long, relative to the test genes in the assay panel, the level of the target genes is overestimated. Thus, for the amplification of FPE tissue RNA subjected to long term storage, especially in case of storage longer than about 7 years, it is important that amplicon lengths, for the target genes and reference genes, be relatively homogeneous, less than about 100 bases, preferably less than about 90 bases, more preferably less than about 80 bases. The lower limit of amplicon size is at least about 45 bases, more preferably at least about 60 bases.

We have discovered that relative levels of particular RNA species present in FPE tissue specimens archived for widely different duration, often many years apart, can be cross-compared by using a reference gene normalization strategy that compensates for the different amounts of RNA degradation that have occurred in the different specimens, as shown in FIG. 4B.

Since the rate of RNA fragmentation in archived FPE tissue has been determined to be proportional to RNA length, optimal correction for the effect of archive storage time requires that the lengths of test gene and reference gene amplicons fall within a narrow range, deviating by not more than about 15%, and preferably by less than about 10%.

(e) Universal Normalization Reference Genes

It is challenging to find genes expressed with little variability between different individual subjects and different tissues. The problem is compounded in cancer tissues where aneuploidy is common, as are both gene and chromosome duplication and/or deletion. The present invention provides a method for identifying universally useful normalization reference genes that avoid such problems.

One class of universally applicable normalization reference genes of the present invention have sequences that are expressed abundantly by reason of redundancy in open reading frames throughout the genome, e.g. human genome. Ideally, the abundance of the expression represents simultaneous transcription from multiple locations throughout the genome. Expressed sequences with this characteristic are relatively insensitive to amplification or deletion of one or a few of the expressed sites, since such amplification or deletion would represent only a minor component of the overall constitutive expression value measured. Similarly, the measured expression represents the average of expression from many sites and therefore will also average and minimize the overall variability of expression.

Candidate sequences for this universal referencing scheme do not need to be structural genes but open reading frames with the required expression pattern (constitutively expressed from multiple sites). Since detection of the expression is by qRT-PCR, any sequence that is conserved (not highly polymorphic) and highly expressed in the genome is potentially useful for this purpose. Such sequences can be readily identified by bioinformatics analysis of expressed sequence databases, and then filtered for map location and tissue expression pattern. Candidate amplicons identified in this way can be functionally tested by qRT-PCR and functionally screened in a representative set of tissues and individual samples, to determine relative variability in expression.

(g) Assay Calibration Sequences

Oligonucleotide sequences that can be used as inert internal assay performance calibration controls are sequences that are not expressed in the human genome. The identification of such reference sequences (here termed internal calibrators) is described in Example 2. In brief, the overall strategy is based on the generation of an initial batch of randomly generated oligonucleotide sequences of approximately 80-100 nucleotide bases. These oligonucleotides are then compared with sequences present in the human genome using publicly available software, such as BLAST, to identify those sequences which show no significant homology. Alternatively, random sequences of shorter oligonucleotides can be generated and compared to sequences present in the human genome, so that sequences with no significant sequence identity can be identified. The short random oligonucleotide sequences that had no significant hits in the human genome are then combined into longer (80-100 bases long) oligonucleotide sequences which can be used as positive internal assay calibration controls, and for a number of other purposes, as described below.

There are at least two advantages for the latter (“bottom-up”) strategy: 1) It improves chances that no sub-string within the amplicon will have a BLAST hit against the human genome, and 2) each of the shorter oligonucleotide sequences (e.g. 21mers) may also serve as a candidate PCR primer that can be used in multiplexed PCR formats.

The internal calibrators of the present invention have multiple potential applications, for example:
(i) qRT-PCR reaction internal positive control to determine if PCR reagents are working in each reaction (well).
(ii) When added as a multiplex component into standard qRT-PCR reactions, these universal “inert” assays can act as internal controls for process calibration. That is, if one of these reactions is added at a standard primer and probe concentration with a known template concentration, the reaction C_{T} should be predictable 100% of the time. When there is a deviation from the expected result, it can be assumed that reaction inhibition or reagent malfunction has occurred and by inference is also affecting the multiplexed reactions to the same degree.
(iii) When multiplexed qRT-PCR is performed, it is desirable to assign one dye label for a control. The internal calibrator can serve this purpose.
(iv) When the RNA sample to be analyzed is spiked with the calibrator complementary RNA, the internal calibrators can serve as positive controls both in qRT-PCR assays and when using hybridization arrays for gene expression analysis.
(v) When the RNA sample is not spiked with complementary RNA, the internal calibrators can serve as negative controls on arrays for gene expression analysis by providing an estimate of non-specific hybridization.
   5. Application of the Results of Gene Expression Profiling

An important aspect of the present invention is to use the measured expression of certain genes in diseased tissue, such as cancer tissues to provide diagnostic and prognostic information. As discussed earlier, for this purpose it is necessary to correct for (normalize away) both differences in the absolute amount of RNA assayed and variability in the quality of the RNA used. Therefore, the assay typically measures and incorporates the expression of certain normalizing or reference genes. Alternatively, normalization can be based on the mean or median signal (C_{T}) of all of the assayed genes or a large subset thereof (global normalization approach).

In order to provide valuable information for treatment decisions, or for classification or various types of cancer, the data obtained by gene expression profiling are typically subjected to statistical analysis. To understand the significance of the expression data, typically a discrimination analysis is performed using a forward stepwise approach. The analysis includes the generation of models for evaluating the gene expression profile, that provide better prognostic information than obtained with any single gene alone.

According to another approach (time-to-event approach), for each gene a Cox Proportional Hazards model (see, e.g. Cox, D. R., and Oakes, D. (1984), Analysis of Survival Data, Chapman and Hall, London, N.Y.) is defined with time to recurrence or death as the dependent variable, and the expression level of the gene as the independent variable. For example, the genes that have a p-value<0.10 in the Cox model are identified. For each gene, the Cox model provides the relative risk (RR) of recurrence or death for a unit change in the expression of the gene. One can choose to partition the patients into subgroups at any threshold value of the measured expression (on the Ct scale), where all patients with expression values above the threshold have higher risk, and all patients with expression values below the threshold have lower risk, or vice versa, depending on whether the gene is an indicator of bad (RR>1.01) or good (RR<1.01) prognosis. Thus, any threshold value will define subgroups of patients with respectively increased or decreased risk.

The implementation of the present invention may be facilitated by the provision of a kit, which includes one or more of the following components: (1) extraction buffer/reagents and protocol; (2) reverse transcription buffer/reagents and protocol; and (3) qPCR buffer/reagents and protocol suitable for performing the method of the present invention. Suitable extraction buffer reagents and protocol are described, for example, in Example 3 below. Suitable reverse transcription buffer/reagents and protocol and qPCR buffer/reagents and protocol are described in the foregoing disclosure and in Example 1. The foregoing disclosure also provides information and directions concerning the design of RT primers and PCR primers and probes. Related software has been discussed, and can be readily adapted to any particular need. The reagents can be conveniently stored, for example, in sealed vials, and the instructions may be attached to (e.g. as a label), or packaged along with the vials, for example as package inserts.

Further details of the invention will be provided in the following non-limiting Examples.

### EXAMPLE 1

Measurement of Gene Expression in Archival Paraffin-Embedded Tissues and Impact of Normalization

Materials and Methods

Tissue Specimens. Archival breast tumor FPE blocks and matching frozen tumor sections were provided by Providence St. Joseph Medical Center, Burbank Calif. Excised tissues were incubated for five to ten hours in 10% neutral-buffered formalin before being alcohol-dehydrated and embedded in paraffin, following standard immunohistology procedures.

RNA extraction procedure. RNA was extracted from three 10 μm FPE sections per each patient case. Paraffin was removed by xylene extraction followed by ethanol wash. RNA was isolated from sectioned tissue blocks using the protocol described in Example 3, with the exception that the MasterPure™ Purification kit (Epicentre, Madison, Wis.) was used for RNA extraction. In the cases of frozen tissue specimens, RNA was extracted using Trizol reagent according to the supplier's instructions (Invitrogen Life Technologies, Carlsbad, Calif.). Residual genomic DNA contamination was assayed by a TaqMan® quantitative PCR assay (no RT control) for β-actin DNA. Samples with measurable residual genomic DNA were re-subjected to DNase I treatment, and assayed again for DNA contamination.

FPE tissue RNA analysis. RNA was quantitated using the RiboGreen® fluorescence method (Molecular Probes, Eugene, Oreg.), and RNA size was analyzed by microcapillary electrophoresis using an Agilent 2100 Bioanalyzer (Agilent Technologies, Palo Alto, Calif.).

TaqMan primer and probe design. For each gene, the appropriate mRNA reference sequence (REFSEQ) accession number was identified and the consensus sequence accessed through the NCBI Entrez nucleotide database. qRT-PCR primers and probes were designed using Primer Express® (Applied Biosystems, Foster City, Calif.) and Primer3 programs. (Rosen and Skaletsky, Methods Mol. Biol. 132:365-386 (2000). Oligonucleotides were supplied by Biosearch Technologies Inc. (Novato, Calif.) and Integrated DNA Technologies (Coralville, Iowa). Amplicon sizes were preferably limited to less than 100 bases in length (see Results). Fluorogenic probes were dual-labeled with 5′-FAM as a reporter and 3′-BHQ-1 as a non-fluorogenic quencher.

Reverse Transcription. Reverse transcription (RT) was carried out using a SuperScript First-Strand Synthesis Kit for qRT-PCR (Invitrogen Corp., Carlsbad, Calif.). Total FPE RNA and pooled gene specific primers were present at 10-50 ng/μl and 100 nM (each) respectively.

TaqMan gene expression profiling. TaqMan reactions were performed in 384 well plates according to instructions of the manufacturer, using Applied Biosystems Prism® 7900HT TaqMan instruments. Expression of each gene was measured either in duplicate 5 μl reactions using cDNA synthesized from 1 ng of total RNA per reaction well, or in single reactions using cDNA synthesized from 2 ng of total RNA, as indicated. Final primer and probe concentrations were 0.9 μM (each primer) and 0.2 μM, respectively. PCR cycling was carried out as follows: 95° C. 10 minutes for one cycle, 95° C. 20 seconds, and 60° C. 45 seconds for 40 cycles. To verify that the qRT-PCR signals derived from RNA rather than genomic DNA, for each gene tested a control identical to the test assay but omitting the RT reaction (no RT control) was included. The threshold cycle for a given amplification curve during qRT-PCR occurs at the point the fluorescent signal from probe cleavage grows beyond a specified fluorescence threshold setting. Test samples with greater initial template exceed the threshold value at earlier amplification cycle numbers than those with lower initial template quantities.

Normalization and data analysis. To compare expression profiles between specimens, normalization based on six reference genes was used to correct for differences arising from variability in RNA quality and total quantity of RNA in each assay. A reference C_{T} (threshold cycle) for each tested specimen was defined as the average measured C_{T} of the six reference genes. Normalized mRNA levels of test genes are define as ΔC_{T}+10, where ΔC_{T}=C_{T} (mean of six reference genes)−C_{T} (test gene).

Statistical analysis. Correlation of gene expression analyses was done using Pearson linear correlation. Cluster analysis was done using 1-Pearson R as the distance metric and single linkage hierarchical clustering.

Results

FPE Tissue RNA Fragmentation Increases with Archive Storage Time.

Capillary electrophoresis analysis of RNA extracted from archival FPE breast cancer specimens shows that the RNA exists largely as fragments of less than 300 bases in length. This is consistent with findings of others (Godfrey et al., supra; Goldsworthy et al., supra). FIG. 2 presents RNA sizing results from specimens archived for substantially different durations. As shown, breast cancer tissue RNA archived for about one year had larger average molecular weight than RNA archived for approximately six or 17 years. (Note detectable 18S RNA at ˜2000 bases in the one year old specimens.) All of these specimens came from one source (Providence Hospital, Burbank, Calif.) and throughout this 17 year period all specimens were fixed using the same formalin fixation protocol (see Materials and Methods for details). This therefore suggests that fragmentation of FPE tissue continues to occur after specimens are dehydrated and embedded in wax.

Results From a 92 Gene Assay: Impact of Amplicon Length on Normalization.

Expression of 92 different genes was profiled (single reaction/well per gene) across 62 different FPE breast cancer specimens that had been archived from one to 17 years. All specimens yielded an adequate quantity of RNA for analysis. The mean and median raw C_{T} for all patients and genes was 33.2 and 32.5, respectively. Raw C_{T} values ranged from 24 to 40 (the latter being the default upper limit PCR cycle number that defines failure to detect a signal as set by the manufacturer).

To be able to compare qRT-PCR data from different tissue specimens, it is necessary to correct for relative differences in input RNA quantity and quality. These differences arise primarily from the variability inherent in processing surgical tissue specimens, including relative mass of tissue and the time between surgery as well as quality and duration of formalin fixation. A secondary consideration is the cumulative variability accrued while processing each sample from RNA extraction through quantitation, reverse transcription to cDNA and PCR. This correction is routinely accomplished by normalizing raw expression values relative to a set of genes that vary little in their median expression among different tissue specimens (“reference genes”).

The observation that RNA continues to degrade with increased archive storage (FIG. 2, above) raised the question whether qRT-PCR signals tend to decay with increased archive storage, and if so, whether normalization to reference genes could compensate for this trend. FIG. 3 shows the mean expression (±SD) relative to the six reference genes for all 92 genes.

Each of the 62 specimens used for the 92 gene study was collected within one of three time ranges, specifically in year 2001, circa 1996, and circa 1985. Each symbol in FIG. 4A represents the average C_{T} across all the tested genes for each of the 62 tested patient specimens. As shown, C_{T} values from the oldest specimens were substantially higher (mean 35.3) than C_{T} values from the newer specimens (mean 31.0). Because the C_{T} scale is log base two, loss of five C_{T} units between year 2001 and 1985 represents a decrease in average qRT-PCR signal of >90%.

Normalization, using a six gene reference set, effectively corrects for this bias (FIG. 4B), flattening the slope of the curve seen in panel 4A and compensating for the loss of qRT-PCR signal that resulted over prolonged storage of FPE specimens. An analysis similar to that shown in FIG. 4B was also carried out on a gene by gene basis (data not shown). In general, individual genes yielded raw data that roughly corresponded to the curve in FIG. 4A prior to normalization, and to FIG. 4B following normalization. However, for 12 genes the age of the block correlated with a rise in average normalized expression. For these 12 genes the average amplicon size was greater (104±15 bases) than the average amplicon size of the other genes in the panel (78±11 bases).

Therefore, when possible, probe and primer sets were redesigned to fit within the relatively narrow range of 70-85 bases. It was found that with the redesigned probe and primer sets normalization corrected for the archive storage-related bias. Thus, optimally, amplicon sizes not only must be limited in length but also the lengths of test gene and reference gene amplicons must be effectively homogeneous.

qRT-PCR is often used as a standard against which to test other gene expression measurement methods, for example DNA array methods (Chuaqui et al. Nature Genetics 32: 509-514 (2002); Rajeevan et al. Methods 25: 443-451 (2001)). Similarly, we sought to compare qRT-PCR-based gene expression profiles from FPE tissue RNA with those from unfixed tissue RNA. For this purpose we identified FPE and frozen samples prepared from the same breast tumor in 1995. The RNA from the frozen tissue remained relatively intact, as indicated by detectable 28S and 18S ribosomal RNA bands. In contrast, much of the RNA from the FPE tissue was smaller than 200 bases in length. The RNAs from the paired FPE and frozen samples were profiled with a 48 gene assay that consisted of 42 test genes and six reference genes. The normalized profiles were not only similar but essentially identical between the two samples for most genes (data not shown). The adjusted Pearson correlation R between FPE and frozen tissue for all tested genes was 91%.

Measured levels of estrogen receptor, progesterone receptor, and HER2 mRNAs were concordant with the levels of the respective proteins as measured by IHC at an independent clinical reference laboratory. Approximately 90% concordance was obtained when qRT-PCR expression results for ER and PR were dichotomized into positive and negative values and compared to ER and PR positive and negative assignments based on IHC (data not shown).

At present, IHC remains the standard gene expression assay that is widely used in diagnostic clinical applications despite its numerous weaknesses which include variation in sensitivity from field to field, dependence on fixation conditions, and lack of calibrated quantitation (Paik et al., J. Natl. Cancer Inst. 94:852-854 (2002)). However, the advantages of qRT-PCR with respect to reproducibility, quantitation, sensitivity, dynamic range, and multi-analyte capability, make this a promising diagnostic technology for immediate future application.

### EXAMPLE 2

Generation of Internal Calibrators

To monitor individual reaction performance and improve the quality control and data normalization process during and after the quantitative qRT-PCR (qRT-PCR) assay for expression profiling, an internal calibration control is desirable to be implemented as one component of multiplexed PCR assays. The purpose of the internal calibration control is to monitor variability in assay performance due such things as variability in assay components or carryover of contaminants in sample extracts. The internal calibrator used for this purpose needs to satisfy the following criteria: (1) it should be an amplicon that satisfies the same length, primer and probe composition, and melting temperature design requirements typically used for the other members of the qRT-PCR assay panel (2) its primers and probes should not interfere by means of sequence interaction with any qRT-PCR assay on human samples (3) sequences of its primers and probes should be absent from the human genome so that it is specific for the synthetic amplicon, and (4) it should exhibit the same efficiency, precision and accuracy in assay performance as the rest of the qRT-PCR assay multiplex panel members.

With the above requirements in mind, a series of internal calibrators were developed for use as positive assay calibration controls. The calibrators were synthetic amplicons of random sequence, 84 nucleotides in length, that were selected because they met assay design requirements and had no significant sequence identity to any sequence in the human genome.

The overall strategy to generate such internal calibrators was started by generating a batch of oligonucleotides of random sequence, each 21 nucleotides in length. These component oligonucleotides were then assembled into random 84-mer oligonucleotides that were compared to the human genome, e.g. using the BLAST software, and the sequences with no significant hits were selected.

1. 1000 random sequences of 21 oligonucleotides were generated.

2. The 1000 oligonucleotides were compared to the human genome using the BLAST software, and those that had no significant hits were selected.

3. The oligonucleotides obtained in step 2 were divided into 4 groups and concatenated into oligos of 84 nucleotides, followed by primer and probe design and further screening.

4. The resulting 84-base oligonucleotides were again compared to the human genome by BLAST, and screened to select the top 16 sequences that had the shortest string of perfect match.

5. Probes and primers were designed for PCR amplification of the 16 oligos and the presence of primer dimers was tested. The final twelve 84-mer oligos that passed the foregoing criteria were selected as internal calibration control sequences.

The selected twelve universal reference sequences are shown in the following Table 2.

TABLE 2

| | | |
|---|---|---|
| Designation | SEQ ID | 5′ => 3′ Internal Calibrator Sequences |

| | | |
|---|---|---|
| IC1 | 1 | CTAGGTCCGTTCATTAGGACAACCCTATCCTAGCGAACTGTCT |
| | | GATCGGCTGAGCATGGGTCGGAAGAGACATCCGCTAACGGT |
| IC2 | 2 | GACGGTCACAGACCTAGAGACGTACTCCCGATCTGTGTCGAT |
| | | GGACGGAATTAGTGCGTACATCTCCCTGGTCGGATTCTAGAG |
| IC3 | 3 | TGTGTCGGGAATGTTGACGTGTCTGACACTGGTGGAATACGC |
| | | AACGCAAGGGCCGCATGTGTCCGCACTAGCGTAGAGTCTTCA |
| IC4 | 4 | ACTTGGCGCGATGATTGACAAAACACCGCGGCCGAAATCCTTT |
| | | GGCGTAGTCCTCGGGTAGTTCGGTCAAAGTTACAGCTGGTT |
| IC5 | 5 | AAATGCGAGGCCGTGGGATCGCGCTGTATGCACCATACCGTA |
| | | AATGTCCAAATACGCGGTCGGGGGTTGTACCGGCAAATGTGC |
| IC6 | 6 | TGGCTGGCTAGGCGAGACATAGGTCAACTGGCTTAGCATACG |
| | | CAGCTAATAGGCTCCGATGCCGAATGCGGATTTAATTCCGGG |
| IC7 | 7 | TCCCATCAGCGCACTCACATACGGATGGGTGGTATCGGGAAG |
| | | TCCCATCAGCGCACTCACATACGGATGGGTGGTATCGGGAAG |
| IC8 | 8 | GAACCGGGACCTGAGCCCAAACGTCAGTCCGGGCTATATCAA |
| | | ATGAGACGCACATAACCGTCCACCCGGCGTATATGCGGATGC |
| IC9 | 9 | CAGTGATGCCGCTACGTCGGTTAATTGGGATTGCGACAGCGT |
| | | CGTCTTGCAGAGCGATACGTTCCAAATTGCGGGTCCTACAGC |
| IC10 | 10 | ACCAGCTCCTAGAGCGAATTGCGCTCAGTGTAACGCCGCTAC |
| | | GCCTCTCGCTCCTGTAAGCCTTATCGGTGGAGGGACTTATAC |
| IC11 | 11 | GACGTCCGCTCCATCAACAGCGACGACCCGCATAATGATCAC |
| | | GGGACGCTAGATAGCTCGAGTTCTCACTCTATGCTCTAGGCC |
| IC12 | 12 | GGCACAAAGAAATCCAGCGTCACTAGGTCAGCTAAGCCGAAA |
| | | AATGTGTGCCTGCGCTCCTCGCCTCATCTCGATGACATACGATG |

Various 21-mer oligonucleotide component sequences used to assemble the 84-mer internal calibrators were selected as potential alternative pairs of PCR priming sequences. These were rechecked to ensure there were no primer cross-hybridizations among the sets. Having alternative PCR primer pair sequences available within each 84-mer calibrator sequence offers the additional advantage of allowing amplicons shorter than 84 nucleotides to be used without any further design work or sequence interaction screening.

### EXAMPLE 3

RNA Extraction from FPE Tissues

RNA is extracted from FPE tissue by the following protocol:
1) Cut 3-6 10 μm sections from the paraffin block;
2) Add 1 ml xylenes and rock 3 minutes;
3) Centrifuge 2 minutes and remove xylene;
4) Add 1 ml fresh xylene and repeat as above 2 more times;
5) Remove all residual xylene from the last incubation;
6) Add 1 ml 100% ethanol and rock 3 minutes;
7) Centrifuge 30 seconds at 14,000 rpm and remove alcohol;
8) Add 1 ml fresh 100% ethanol and repeat 2 more times;
9) Remove all residual alcohol and add 300 μl proteinase K in digestion buffer.

Digestion buffer formula:
4M urea 10 mM TrisCl pH 7.5, 0.5-1.0% sodium lauroyl sarcosine and 330 μg/ml proteinase K.

Alternatively, 1M ethanolamine or 1M Guanidine isothiocyanate, may be substituted for urea to yield similar quality and quantity of RNA.
10) Incubate tissue sections in proteinase K solution for 90 minutes at 65° C. with constant shaking at 850 rpm (with Eppendorf Thermomixer);
11) Add 150 μl of 7.5 M NH₄OAc and vortex 10 seconds. Centrifuge for 10 minutes at 14K rpm. Pippette the supernatant to a fresh tube avoiding the white and sometimes clear pellet at the bottom. This will remove the proteinase K and other proteins in solution during the lysis;
12) Add an equal volume of isopropyl alcohol to the harvested supernatant and rock 5 minutes before centrifugation at 4° C.;
13) A white RNA pellet should be visible at the bottom side of the tube;
14) Wash the pellet with 1 ml 80% ethanol, quick centrifuge and remove ethanol, repeat;
15) Air dry pellet and resuspend in nuclease free water.

While the present invention has been described with reference to what are considered to be the specific embodiments, it is to be understood that the invention is not limited to such embodiments. To the contrary, the invention is intended to cover various modifications and equivalents included within the spirit and scope of the appended claims. For example, while the disclosure focuses on the gene expression profiling of tissue samples obtained from cancer, in particular FPET samples, the method of the present invention is equally suitable for determining the gene expression profile of any biological sample, whether normal or diseased. In particular, the method of the present invention is suitable for the expression profiling of all biological samples containing fragmented and/or chemically processed (modified) RNA, including aged, preserved and processed samples, such as forensic samples and pathology samples.

Although the methods of the present invention have been illustrated by qRT-PCR of the TaqMan® format, which requires two PCR primers and one intervening, dually labeled reporter probe, it is not so limited. Alternative assay formats are compatible with the optimized analytical assay of the present invention, including, without limitation, probe and primer formats adapted to the LightCycler qRT-PCR instrument, Scorpion™ Probes for qRT-PCR, MGB®-modified probes for qRT-PCR, SNPdragon™ probes for qRT-PCR, Molecular Beacon probes, extension primers designed for detection by MALDI-TOF Mass Spectrometry and other like modifications of the qRT-PCR assay format. All such and similar modifications, which serve to enhance, customize or modify of the qRT-PCR-based assays of the present invention, will be apparent to those skilled in the art, and are specifically within the scope of the present invention.

All references cited throughout the disclosure are hereby expressly incorporated by reference.

Although the invention is illustrated by reference to certain embodiments, it is not so limited. One of ordinary skill in the art will appreciate that certain modifications and variations are possible, and will provide essentially the same result in essentially the same way. All such modifications are variations are within the scope of the invention claimed herein.

1. A method for determining RNA expression profile in a tissue sample comprising a plurality of RNA species, by quantitative reverse transcription polymerase chain reaction (qRT-PCR), comprising the steps of:
(a) extracting RNA from said sample under conditions that provide a maximum representation of all transcribed RNA species present in said tissue sample;
(b) treating the RNA obtained with a reverse transcription reaction mixture comprising a plurality of gene-specific oligonucleotides corresponding to at least a subset of said RNA species, dNTPs and a reverse transcriptase, under conditions allowing transcription of said RNA into complementary DNA (cDNA);
(c) quantitatively detecting each cDNA transcript, wherein steps (b) and (c) are performed in separate reactions.

2. The method of claim 1 wherein said cDNA obtained in step (b) is amplified before performing step (c).

3. The method of claim 2 wherein amplification is performed by polymerase chain reaction (PCR), in the presence of a set of forward and reverse primers to generate an amplicon for each cDNA transcript.

4. The method of claim 3 wherein at least part of the gene-specific oligonucleotides used in step (b) serve as reverse primers in the PCR amplification step.

5. The method of claim 1 wherein said tissue is aged, preserved or processed tissue, comprising fragmented or chemically modified RNA.

6. The method of claim 4 wherein said tissue is human tissue.

7. The method of claim 6 wherein said tissue is a frozen or fixed, wax-embedded tissue.

8. The method of claim 7 wherein in step (b) said reverse transcription mixture comprises gene specific oligonucleotides for at least about 10 RNA species.

9. The method of claim 7 wherein in step (b) said reverse transcription mixture comprises gene specific oligonucleotides for at least about 15 RNA species.

10. The method of claim 7 wherein in step (b) said reverse transcription mixture comprises gene specific oligonucleotides for at least about 90 RNA species.

11. The method of claim 7 wherein in step (b) said reverse transcription mixture comprises gene specific oligonucleotides for at least about 400 RNA species

12. The method of claim 7 wherein in step (b) said reverse transcription mixture comprises gene specific oligonucleotides for at least about 800 RNA species.

13. The method of claim 7 wherein in step (b) said reverse transcription mixture comprises gene specific oligonucleotides for at least about 1600 RNA species.

14. The method of claim 7 wherein said reverse transcription mixture further comprises a plurality of random oligonucleotides.

15. The method of claim 14 wherein said random oligonucleotides are 6- to 10-nucleotides long.

16. The method of claim 14 wherein said random oligonucleotides are 6-nucleotides long.

17. The method of claim 14 wherein said random oligonucleotides are 8 nucleotides long.

18. The method of claim 14 wherein said random oligonucleotides are 9 nucleotides long.

19. The method of claim 7 wherein in the reverse transcriptase step (b) or the PCR amplification step, or both steps, the number of oligonucleotides susceptible for self-priming or cross-priming is minimized.

20. The method of claim 19 wherein self-priming or cross-priming is minimized by a computer algorithm.

21. The method of claim 7 wherein said reverse transcription mixture comprises RNA of at least one normalization reference sequence.

22. The method of claim 21 wherein the reverse transcription mixture in step (b) comprises RNA of about 5 to 10 normalization reference sequences.

23. The method of claim 7 wherein each qRT-PCR reaction includes at least one internal calibration reference sequence.

24. The method of claim 23 wherein one or more of said internal calibration reference sequences include sequences which have no significant homology to any sequence in the human genome.

25. The method of claim 7 wherein said tissue sample is a frozen or formalin fixed, paraffin-embedded (FPE) biopsy sample from a tumor.

26. The method of claim 25 wherein said tumor is cancer.

27. The method of claim 25 wherein said cancer is selected from the group consisting of breast cancer, colon cancer, lung cancer, prostate cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, and brain cancer.

28. The method of claim 25 wherein said cancer tissue comprises fragmented RNA.

29. The method of claim 28 wherein gene target amplicons are less than about 100 nucleotides long.

30. The method of claim 28 wherein the gene target amplicons are less than about 90 nucleotides long.

31. The method of claim 28 wherein the gene target amplicons are less than about 80 nucleotides long.

32. The method of claim 28 wherein the difference between the length of the amplicons of the target genes and the normalization reference genes is not more than about 15%.

33. The method of claim 28 wherein the difference between the length of the amplicons of the target genes and the normalization reference genes is less than about 10%.

34. The method of claim 21 wherein the gene expression levels are normalized relative to said normalization reference sequence or sequences.

35. The method of claim 34 wherein the gene expression levels are normalized relative to one or more normalization reference genes selected from the group consisting of β-ACTIN, CYP1, GUS, RPLPO, TBP, GAPDH, and TFRC.

36. The method of claim 35 wherein the gene expression levels are corrected relative to one or more universal internal calibration reference sequences.

37. The method of claim 26 further comprising the step of identifying one or more genes the expression of which is correlated with the presence or likelihood of recurrence of said cancer.

38. The method of claim 26 further comprising the step of subjecting the gene expression profile to statistical analysis.

39. The method of claim 38 further comprising the step of preparing a report for a subject whose cancer tissue is analyzed.

40. The method of claim 39 wherein said report includes a statement of likelihood of survival without cancer recurrence, or likelihood of response to a certain chemotherapeutic drug or drug set.

41. A kit comprising one or more of (1) extraction buffer/reagents and protocol; (2) reverse transcription buffer/reagents and protocol; and (3) qPCR buffer/reagents and protocol suitable for performing the method of any one of claims 1-3.

42. The kit of claim 41 further comprising a data retrieval and analysis software.

43. The kit of claim 41 wherein component (2) includes pre-designed primers.

44. The kit of claim 41 wherein component (3) includes pre-designed PCR probes and primers.
